(19) United States
(12) Patent Application Publication
(10) Pub. No.: US 2008/0089949 A1
(43) Pub. Date: Apr. 17, 2008

(54) Method for treating cancer using oral arsenic trioxide

(76) Inventor: Yok-Lam Kwong, Hong Kong (HK)

Correspondence Address: COOPER & DUNHAM, LLP, 1185 AVENUE OF THE AMERICAS, NEW YORK, NY, 10036, US

(21) Appl. No.: 11/549,347

(22) Filed: Oct. 13, 2006

(51) Int. Cl. A61K 33/36

(52) U.S. Cl. 424/623

(57) ABSTRACT

The invention provides a method for treating cancers that are dependent on cyclin D1 for proliferation, survival, metastasis and differentiation, involving administering effective amount of arsenic trioxide to an affected patient.

### FIELD OF THE INVENTION

This invention relates to methods of treating cancer by affecting expression, translation, and biological activity of cyclin D1 using arsenic trioxide.

### REFERENCES

Several publications are referenced herein by Arabic numerals with parenthesis. Full citations for these references may be found at the end of the specification immediately preceding the claims. These references are incorporated herein by reference.

### BACKGROUND OF THE INVENTION

Cyclin D1 is a D-type cyclin critically involved in the control of cell cycle. It assembles with its catalytic partners cyclin-dependent kinase 4 (CDK4) and CDK6 to form an active holoenzyme complex, which controls G1 progression and G1/S transition.¹ The active holoenzyme complex phosphorylates the retinoblastoma protein RB. Phosphorylated RB releases the E2F family of transcription factors from inhibition, enabling E2Fs to coordinately regulate genes necessary for DNA replication and hence progression into S phase.² Over-expression of cyclin D1 is demonstrable in many cancers, including cancers of the digestive tract, cancers of the female genital tract, and malignant lymphomas.

Owing to its important influence on the cell cycle, cyclin D1 expression is carefully regulated. Cyclin D1 gene mRNA and transcription appears to be constant through the cell cycle.³ However, a decline in cyclin D1 level occurs during S phase, which has been attributed to its increased proteasomal degradation.^{∝}Cyclin D1 phosphorylation at a threonine residue 286 (Thr-286) positively regulates its proteasomal degradation.⁵ Thr-286 phosphorylation is mediated by glycogen synthase kinase-3β (GSK-3β).⁶ In addition to targeting cyclin D1 to proteosomes, GSK-3β-induced Thr-286 phosphorylation also promotes cyclin D1 nuclear export, by increasing the binding of cyclin D1 to a nuclear exportin CRM1.⁷ Recently, it has been shown that the IkappaB kinase (IKK) alpha, IKKα, associates with and phosphorylates cyclin D1 also at Thr-286, thereby participating in the subcellular localization and turnover of cyclin D1.⁸

Mantle cell lymphoma (MCL) is a well-defined subtype of B cell lymphoma in the World Health Organization classification,⁹ and accounts for approximately 3-10% of all non-Hodgkin lymphomas.¹⁰ The chromosomal aberration t(11;14)(q13;q32) can be found in practically all cases of MCL.⁹ The translocation results in juxtaposition of the immunoglobulin heavy chain joining region on chromosome 14 to the cyclin D1 gene on chromosome 11.¹¹ The molecular consequence of the translocation is to place cyclin D1 under the control of the immunoglobulin heavy chain gene enhancer,¹² leading to over-expression of the cyclin D1 protein. Therefore, MCL is an important prototype of cancer formation due to over-expression of cyclin D1. It is a very useful model in the investigation of the contribution of cyclin D1 to cancer formation. Furthermore, it provides a pertinent model for the study of therapeutic agents in the treatment of cancers over-expression cyclin D1.

Although MCL accounts for approximately 3-8% of B-cell lymphomas, it is difficult to manage.¹³ Initial treatment with rituximab plus combination chemotherapy or purine analogues results in complete remission (CR) rates varying from 34-87%.¹⁴⁻⁶ However, relapses occur in most patients with prolonged follow up. Treatment options for relapsed patients are limited. Several novel approaches have been adopted, including the use of the proteasome inhibitor bortezomib,^{17,18} thalidomide¹⁹ and the mammalian target of rapamycin (mTOR) inhibitor temsirolimus.²⁰ The overall response (OR) rates of these agents varied from 38-81%, but the CR rate was only 3-31%. Therefore, there is an urgent need to define novel treatment strategies for MCL.

Arsenic trioxide (As₂O₃) is a standard treatment for acute promyelocytic leukemia (APL).²¹ Additionally, it has shown clinical activity in other hematologic malignancies, notably lymphomas.²¹ The inventors have invented an oral arsenic trioxide preparation for the treatment of patients with blood cancers.²² Furthermore, the inventors have shown that oral As₂O₃ is highly efficacious for relapsed acute promyelocytic leukemia.²³ Moreover, the inventors have shown that oral As₂O₃, causes a smaller prolongation of QT intervals, and therefore may provide a much safer drug for treating leukemia.²⁴

Since cyclin D1 plays a pivotal role in the pathogenesis of MCL, the inventors tested the hypothesis that As₂O₃ might target cyclin D1 in MCL. The inventors also used MCL as a model for the therapeutic use of As₂O₃ in targeting cancers that over-expressed cyclin D1. Finally, the inventors also tested clinically the therapeutic use of As₂O₃ in patients with terminal or refractory MCL.

It is an object of this invention to provide agents and methods for degrading cyclin D1 in cancers. It is also an object of this invention to provide agents of As₂O₃ in the treatment of cancers related to cyclin D1 over-expression. Another object of this invention is to provide agents and the in vivo concentration of As₂O₃ needed to produce therapeutic effects in cancers over-expressing cyclin D1. This invention further provides methods, strategies, doses, and dosing schedule in the application of As₂O₃ in the clinical treatment of cancers over-expressing cyclin D1.

### SUMMARY OF THE INVENTION

The inventors have discovered that As₂O₃ suppresses cyclin D1. The inventors also discovered that As₂O₃ initiated down-regulation of cyclin D1 by activating GSK-3β, which phosphorylates cyclin D1. The inventors also discovered that the IKKb was activated, leading to phosphorylation of cyclin D1. The inventors further discovered that phosphorylated cyclin D1 was ubiquitinated. The inventors then showed that ubiquitinated cyclin D1 was degraded in the proteasome.

### BRIEF DESCRIPTION OF DRAWINGS

These and other objects, features and advantages will become apparent upon reviewing the following detailed description of the preferred embodiments, in conjunction with the attached drawings, which are briefly described below.

FIG. 1 shows As₂O₃ induced apoptosis in MCL cells. A. MTT test of Jeko-1 and Granta-519 cells treated for 72 hours with As2O3. There was a dose and time dependent suppression of cellular proliferation. Viability significantly decreased at or above 1 μM As₂O₃ as compared with baseline (one-way ANOVA with Dunnett's post-tests, p<0.05) (triplicate experiments). B. Significant increase in apoptotic cells after As₂O₃ treatment. #: apoptotic cells that were annexin V positive and popidium iodide negative. C. Activation of caspase 3 by As₂O₃ treatment. Cleaved caspase 3 were detectable four hours after As₂O₃ treatment.

FIG. 2 shows down-regulation of cyclin D1 by As₂O₃ treatment. A. As₂O₃ (4 μM) induced a time dependent down-regulation of cyclin D1 in Jeko-1 and Granta-519 cells. Triplicate experiments and a representative Western blot demonstrate significant decrease in cyclin D1 level after 2 hours (one-way ANOVA with Dunnett's post-tests, p<0.05) B. As₂O₃ (treatment for 8 hours) induced a dose dependent down-regulation of cyclin D1 in Jeko-1 and Granta-519 cells. Triplicate experiments and a representative Western blot demonstrate significant decrease in cyclin D1 level at or above 2 μM (one-way ANOVA with Dunnett's post-tests, p<0.05). C. Semi-quantitative polymerase chain reaction showing that cyclin D1 gene transcription was unaffected by As₂O₃ treatment.

FIG. 3 shows dephosphorylation of retinoblastoma (RB) by As₂O₃ treatment in MCL lines. As₂O₃ treatment resulted in dephosphorylation of RB (significant decrease of phosphor-Rb Ser-795 at or more that 8 hours of As₂O₃ treatment, triplicate experiments, one-way ANOVA with Dunnett's post-tests, p<0.05). A representative Western blot was shown.

FIG. 4 shows As₂O₃ treatment induced phosphorylation of cyclin D1 and GSK-3. A. Cell lysates immunoblotted with anti-phospho-cyclin D1 (Thr-286). As₂O₃ treatment led to significantly increased phosphor-cyclin D1 (triplicate experiments, one-way ANOVA with Dunnett's post-tests, p<0.05). B. Cell lysates immunoblotted with anti-phospho-cyclin GSK-3β (Try-216). As₂O₃ treatment led to significantly increased phosphor-GSK-3β (triplicate experiments, one-way ANOVA with Dunnett's post-tests, p<0.05). C. Pre-incubation with 6-bromoindirubin-3′-oxime (BIO; 10 μM) before As₂O₃ treatment (4 μM, 8 hour, 37° C.) prevented cyclin D1 down-regulation, showing that GSK-3b was involved. Result a significant reduction of cyclin D1 as compared with control (triplicate experiments, one-way ANOVA with Dunnett's post-tests, p<0.05).

FIG. 5 shows that IKK was involved in As2O3-induced down-regulation of cyclin D1. A. As₂O₃ treatment (4 μM for 2 hours) led to a significant increase in phosphor-IKKα/β (Ser-176/180) (triplicate experiments, one-way ANOVA with Dunnett's post-tests, p<0.05). B. Pre-incubation with the IKK inhibitor BMS (10 mM, 30 minutes) successfully prevented As₂O₃-induced cyclin D1 down-regulation (triplicate experiments, one-way ANOVA with Dunnett's post-tests, p<0.05). C. Cells were treated with As₂O₃ (4 μM, 2 hours), followed by lysis and immunoprecipitation with an anti-IKKα/β or anti-cyclin D1 antibody. The immunoprecipitates and the crude lysates were immunoblotted with anti-cyclin D1 and anti-IKKα/β antisera. As₂O₃ induced an increase in binding between IKKα/β and cyclin D1.

FIG. 6 is a As₂O₃ induced ubiquitination of cyclin D1 in MCL. A. Cell lysates were immunoprecipitation with anti ubiquitin (Ub) or anti-cyclin D1 antibody. The immunoprecipitates and the crude lysates were immunoblotted with anti-cyclin D1 and anti-ubiquitin antisera. As2O3 induced a significant increase in binding between cyclin D1 and ubiquitin (increase in ubiquitination from 30 minutes to 2 hours after As₂O₃ treatment as compared to the baseline, triplicate experiments, one-way ANOVA with Dunnett's post-tests, p<0.05). A representative Western blot was shown.

FIG. 7 shows As₂O₃-induced cyclin D1 degradation involved the proteasome but not the lysosome in MCL. A. Pre-incubation with the proteasome inhibitors MG132 (MG, 30 μM), bortezomib (bort, 10 μg/ml) and lactacystin (lact, 10 μM) successfully prevented As2O3 induced cyclin D1 degradation. B. Pre-incubation with the lysosomal inhibitor ammonium chloride (NH₄Cl, 2.5 mM) was ineffective in preventing As₂O₃-induced cyclin D1 degradation.

FIG. 8 is a schematic diagram showing the proposal degradation of cyclin D1 mediated by As₂O₃.

FIG. 9 is a response of mantle cell lymphoma to oral arsenic trioxide (As₂O₃). A. Case 6, with bilateral orbital infiltration at relapse (A1) that completely resolved (A2) after 4 months of oral-As₂O₃ treatment and ascorbic acids, AA. B. Case 8, relapsing as leukemic phase and massive splenomegaly (B1), who achieved a partial remission after 8 months of treatment with oral-As₂O₃ and AA (B2). C. Case 8, with dense marrow infiltration (C1) that resolved (C2) after 8 months of treatment with oral-As₂O₃ and AA.

### DETAILED DESCRIPTION OF THE PREFERRED EMBODIMENTS

Reference will now be made in detail to the presently preferred embodiments of the invention, which, together with the examples following the detailed description, serve to explain the principles of the invention.

The inventors discovered that As₂O₃ induced apoptosis in MCL lines at 2-4 μM, which was within the plasma levels achieved after As₂O₃ therapy. As₂O₃ induced a dose and time dependent suppression of cyclin D1. The suppression of cyclin D1 restored RB to a hypophosphorylated state, in parallel with a change in cell cycle. These biologic changes were consistent with the apoptosis observed upon As₂O₃ treatment.

The inventors further showed that the down-regulation of cyclin D1 mediated by As₂O₃ occurred at a post-transcriptional level. This might be expected, as cyclin D1 is under the transcriptional control of the immunoglobulin heavy chain gene enhancer in MCL, which is unlikely to be affected by As₂O₃. Furthermore, in physiologic conditions, the control of cyclin D1 during the cell cycle is also mediated in part via alteration in the stability of cyclin D1.⁷ This process is controlled by phosphorylation of cyclin D1 at Thr-286, a process mediated by GSK-3β.⁸⁻¹¹ GSK-3β is itself tightly regulated. Mitogens inactivate GSK-3β by a pathway involving Ras, phosphatidylinositol 3 kinase (PI3K), and protein kinaseB/Akt.^{25,26} Ras activates PI3K, which in turn activates Akt. Akt inactivates GSK-3β by phosphorylating it at serine residue 9.²⁷ This removes the inhibition of GSK-3β on cyclin D1, allowing cyclin D1 to accumulate and thus activate cell cycling.

On the other hand, GSK-3β can also be activated by phosphorylation at a tyrosine residue 216 (Try-216) in the kinase domain.²⁸ Little is known, though, of the physiologic mechanisms controlling GSK-3β phosphorylation at Tyr-216. There is some evidence that GSK-3β might autophorphorylate.²⁹ In D. discoideum, the tyrosine kinase ZAK1 phosphorylates GSK-3β in response to cAMP.³⁰ In mammalian cells, the tyrosine kinases Fyn,³¹ Csk³² and Pyk2^{33,34} have been implicated in GSK-3β phosphorylation at Tyr-216. Therefore, an important novel observation in this study is the As₂O₃-mediated increase of GSK-3β Try-216 phosphorylation. How an inorganic molecule As₂O₃ might enhance GSK-3β phosphorylation remains to be defined. However, it has been shown that increases in calcium may lead to enhanced GSK-3β phosphorylation, via activation of the calcium sensitive kinase Pyk2.³⁵ Whether As₂O₃ acts through a similar mechanism will have to be investigated. Nevertheless, the end result of As₂O₃-mediated increase in GSK-3β Try-216 phosphorylation is the increase in cyclin D1 Thr-286 phosphorylation, a key step in its degradation.

Another recently defined mechanism of regulating cyclin D1 is the IKK system. The IKK complex is the major regulatory component in the NK-κB pathway. It comprises the catalytic subunits IKKα and IKKβ, and a regulatory subunit IKKγ/NEMO.³⁶ Interestingly, IKKα has been shown recently to phosphorylate cyclin D1 at Thr-286, the same site targeted by GSK-3b. IKKα needs to be activated by phosphorylation at a serine residue 176 (Ser-176) before participating in the regulation of NF-κB by phosphorylating IκB.³⁸ IKKα Ser-176 phosphorylation is mediated by NK-κB inducing kinase (NIK).³⁶ Hence, the finding of As₂O₃-induced increase in IKK phosphorylation is another important original observation. Furthermore, As₂O₃-mediated an increase in physical interaction between IKK and cyclin D1, as shown in immunoprecipitation experiments. Finally, an IKK specific inhibitor BMS-345541³⁷ alleviated As₂O₃-induced cyclin D1 down-regulation. Taken together, these results indicated that IKK was also an effector of As₂O₃ treatment. The mechanism by which As₂O₃ increases IKK phosphorylation is unclear. However, NIK is activated by a host of stimuli, including tumor necrosis factor and interleukin-1.³⁸ The potential interaction of As₂O₃ with these signaling molecules requires future studies.

The inventors further showed that As₂O₃-mediated cyclin D1 Thr-286 phosphorylation increased its ubiquitination. Moreover, the time course of ubiquitination was commensurate with the timing of the biologic functions of As₂O₃ on the MCL lines. After As₂O₃ treatment, increased ubiquitination was first detected at 30 minutes and continued to increase. At two hours, significant down-regulation of cyclin D1 was first observed, which was associated with a parallel hypophosphorylation of RB. Finally, significant activation of caspase 3 was observed at four hours. These sequence of events were consistent with cyclin D1 down-regulation initiated by Thr-286 phosphorylation.

Cyclin D1 is a cytosolic and nuclear protein. Therefore, polyubiquitination is involved, which targets the protein to degradation in proteasomes. Indeed, we showed that inhibition of proteasomes successfully prevented As₂O₃-induced down-regulation of cyclin D1. On the other hand, inhibition of lysosomes, the site of degradation of monoubiquitinated proteins,³⁹ did not interfere with As₂O₃-induced down-regulation of cyclin D1. These results confirm that As₂O₃ down-regulated cyclin D1 by promoting its proteasomal degradation.

The capability of As₂O₃ in augmenting proteasomal degradation of cyclin D1 is reminiscent of its action on another fusion oncoprotein PML-RARA in APL. As₂O₃ enhances the conjugation of a ubiquitin-related peptide SUMO-1 to the PML part of the PML-RARA protein.⁴⁰ This directs PML-RARA to nuclear bodies, which are nuclear matrix domains containing 11S proteasome constituents recruited by As₂O₃ treatment. In this way, As₂O₃ triggers proteasome-dependent degradation of SUMO-conjugated PML-RARA.⁴¹ Therefore, As₂O₃ may act together with component of the proteasomal system to effect degradation of target proteins. Findings of the current study corroborate with this proposition.

The inventors have clearly shown that As₂O₃ suppresses MCL cell growth by targeting cyclin D1. Furthermore, there are a number of important ramifications arising from this study that will form the lead for further investigations. As₂O₃ appears to be capable of inducing the phosphorylation of not only GSK-3β, but also IKK. The issues of whether this is mediated by different mechanisms or a common pathway, and the possibility that As₂O₃ might mediate phosphorylation of other biologically important molecules, will warrant exploration. Finally, cyclin D1 over-expression is pathogenetically important in a vast diversity of cancers. It is important to determine if As₂O₃ also targets cyclin D1 in these cancers, and is therefore of therapeutic potential.

Based on these observations, the inventors used oral-As₂O₃ in the treatment of 14 patients with refractory or relapsed MCL, which over-expressed cyclin D1. The inventors observed an overall response in 9 patients (64%). Four patients achieved complete remission, two patients complete remission unconfirmed, and three patients with partial remissions. These results were very good, given that these patients had refractory or relapsed disease. These clinical observations obtained by the inventors are a direct in vivo proof of the inventors' observations in their experimental system.

Taken together, the inventors have discovered several novel findings. The inventors have discovered that As₂O₃ decreased cyclin D1. The inventors further discovered that the decrease in cyclin D1 was post-transcriptional. The inventors moreover discovered that As₂O₃ induced GSK-3β and IKK activation and hence phosphorylation of cyclin D1. The inventors then showed that phosphorylated cyclin D1 was degraded in the proteasome. Finally, the inventors have made the novel observation that oral As₂O₃ induced a high response rate clinically in patients with refractory or relapsed MCL, a cancer that over-expressed cyclin D1.

The present invention of using oral arsenic trioxide in suppressing cyclin D1 is an important paradigm applicable to the treatment of cancers that are dependent on cyclin D1 for proliferation, survival, metastasis and differentiation.

The following delivery systems, which employ a number of routinely used pharmaceutical carriers, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

### EXAMPLE 1 OF As₂O₃ IN MCL BY TARGETING CYCLIN D1

Call lines. The MCL lines Jeko-1 and Granta-519 were obtained from German Collection of Microorganisms and Cell Cultures (ACC 553 and ACC 342, Braunschweig, Germany). Jeko-1 cells were cultured in RPMI 1640 with 20% fetal bovine serum (FBS), and Granta-519 cells in DMEM with 10% FBS; both with 50 units/ml penicillin and 50 μg/ml streptomycin, at 5% CO₂.

### EXAMPLE 2 OF As₂O₃ IN MCL BY TARGETING CYCLIN D1

Reagents and antibodies. Reagents and antibodies used included cell culture reagents (Invitrogen, Carlsbad, Calif., USA); kinase inhibitors and their inactive analogues (Calbiochem, Darmstadt, Germany); antiserum to phospho-GSK3 (tyrosine 216, Try-216) (Upstate, Lake Placid, N.Y., USA); antisera to cyclin D1, phospho-cyclin D1 (Thr-286), GSK3β, phospho-GSK3β (Tyr-216), IκB kinase (IKK) α/β, phospho-IKKα/β (serine 176/180, Ser-176/180), RB and phospho-RB (serine 795, Ser-795), caspase-3 and β-actin (Cell Signaling Technology, Beverly, Mass., USA); protein G-agarose (Upstate); ECL kit (Amersham, Piscataway, N.J., USA); cell proliferation kit I (MTT) (Roche Applied Science, Indianapolis, Ind., USA); annexin V-FITC Kit (Beckman Coulter, Fullerton, Calif., USA); and RNeasy Kit and One-Step RT-PCR Kit (Qiagen, Valencia, Calif., USA).

### EXAMPLE 3 OF As₂O₃ IN MCL BY TARGETING CYCLIN D1

Cell viability assays. Cells were seeded on 96-well microplates at 2×10⁴/well in 100 ml growth medium containing different concentration of As₂O₃ as indicated at 37° C. for 72 hours. MTT labeling reagent (10 μl, 5 mg/ml) (Roche Applied Science, Indianapolis, Ind., USA) was added to each well at 37° C. for 4 hours, followed by 100 μl solubilization at 37° C. overnight. Solubilized fomarzan crystals were quantified spectophotometrically at 590 nm with a microplate ELISA reader.

### EXAMPLE 4 OF As₂O₃ IN MCL BY TARGETING CYCLIN D1

Apoptosis assay. Cells were seeded at 1×10⁶/ml in different concentrations of As₂O₃ as indicated at 37° C. for 24 hours, harvested, rinsed in ice-cold phosphate buffered saline (PBS), and resuspended in 500 □1 binding buffer containing annexin V-FITC and propidium iodide (PI) (Beckman Coulter, Fullerton, Calif., USA) for 20 minutes on ice. The percentages of apoptotic cells (annexin-V positive, PI negative) were determined on a flow cytometer (Epics, Beckman Coulter) with appropriate color compensation.

Cell Cycle Analysis. Cells were seeded at 1×10⁵/ml in different concentrations of As₂O₃ as indicated at 37° C. for 8 hours, harvested, washed in ice-cold PBS, resuspended in 500 μl PBS, stained with PI for 10 minutes on ice. Cell cycle was determined by flow cytometry (Epics, Beckman Coulter).

### EXAMPLE 5 OF As₂O₃ IN MCL BY TARGETING CYCLIN D1

Semi-quantitative reverse transcription polymerase chain reaction (RT-PCR) for cyclin D1. Cells were seeded at a density of 1×10⁶/ml in different concentrations of As₂O₃ at 37° C. for 8 hours, washed with PBS buffer and lysed with RTL buffer. RNA was extracted with an RNeasy Kit, followed by cDNA synthesis and a 30-cycle PCR with a One-Step RT-PCR Kit with the forward primer 5′-CTG GCC ATG AAC TAC CTG GA-3′ and the reverse primer 5′-GTC ACA CTT GAT CAC TCT GG-3′. Cycling conditions were denaturation (1 minute at 94° C., first cycle 5 minutes), annealing (2 minutes at 50° C.) and extension (3 minutes at 72° C., last cycle 10 minutes),

### EXAMPLE 6 OF As₂O₃ IN MCL BY TARGETING CYCLIN D1

Western Blotting Analysis. Cells were seeded at a density of 1×10⁶/ml overnight. Where applicable, cells were pre-treated with various inhibitors for 30 minutes, and then incubated with 4 μM As₂O₃ for different time periods as indicated. Cells were lysed in lysis buffer (50 mM Tris-HCl, 100 mM NaCl, 5 mM EDTA, 40 mM NaP₂O₇, pH 7.5, 1% Triton X-100, 4 μg/ml aprotinin, 1 mM dithiothreitol, 200 μM Na₃VO₄, 0.7 μg/ml pepstatin, 100 μM phenylmethylsulfonyl fluoride, and 2 μg/ml leupeptin). Clarified lysates were resolved on 12% SDS-phenylmethylsulfonyl fluoride and transferred to nitrocellulose membranes. The membranes were blocked with 5% non-fat milk, washed, incubated with the appropriate antibodies followed by horseradish peroxidase-conjugated secondary antisera. Immuno-reactive bands were visualized by chemiluminescence with the ECL kit, detected on X-ray films and quantified by densitometric scanning (Eagle Eye II still video system, Stratagene, La Jolla, Calif., USA).

### EXAMPLE 7 OF As₂O₃ IN MCL BY TARGETING CYCLIN D1

Coimmunoprecipitation Assays. Cells were seeded at 1×10⁶/ml overnight treated with 4 μM As₂O₃ at 37° C. for different time periods as indicated, and lysed in lysis buffer. Cell lysates were incubated with an anti-cyclin D1, anti-ubiquitin, anti-calpain 2 or anti-IKKα/β antibodies (4 μg/sample) at 4° C. for 1 hour, followed by incubation with 30 μl of protein G-agarose (50% slurry) at 4° C. for another 2 hour. Immunoprecipitates were washed four times with 400 μl lysis buffer, resuspended in 50 μl lysis buffer and 10 ml 6× sample buffer and boiled for 5 minutes. Immunoprecipitates were then analysed by Western blot analysis.

### RESULTS OF EXAMPLE 1-7 OF As₂O₃ IN MCL BY TARGETING CYCLIN D1

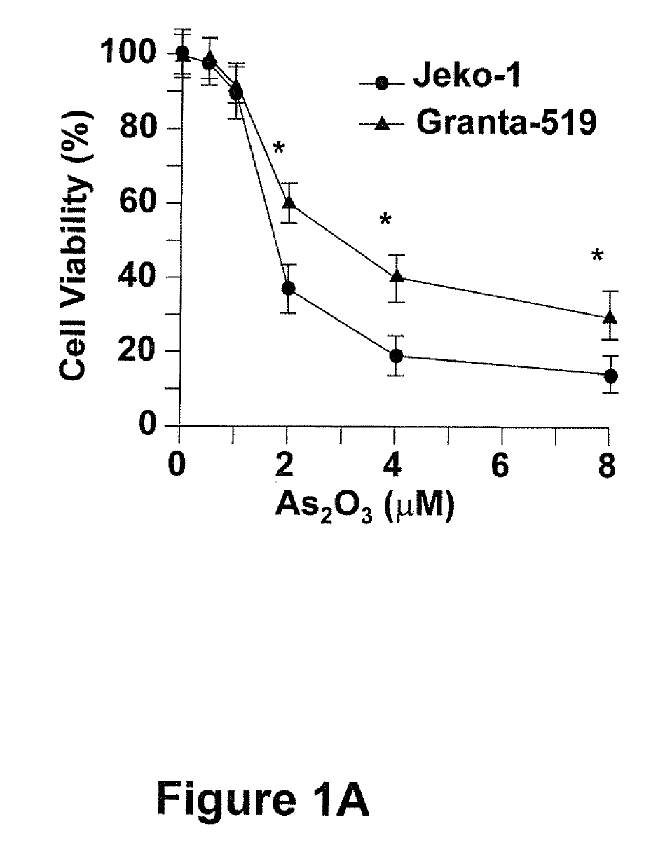

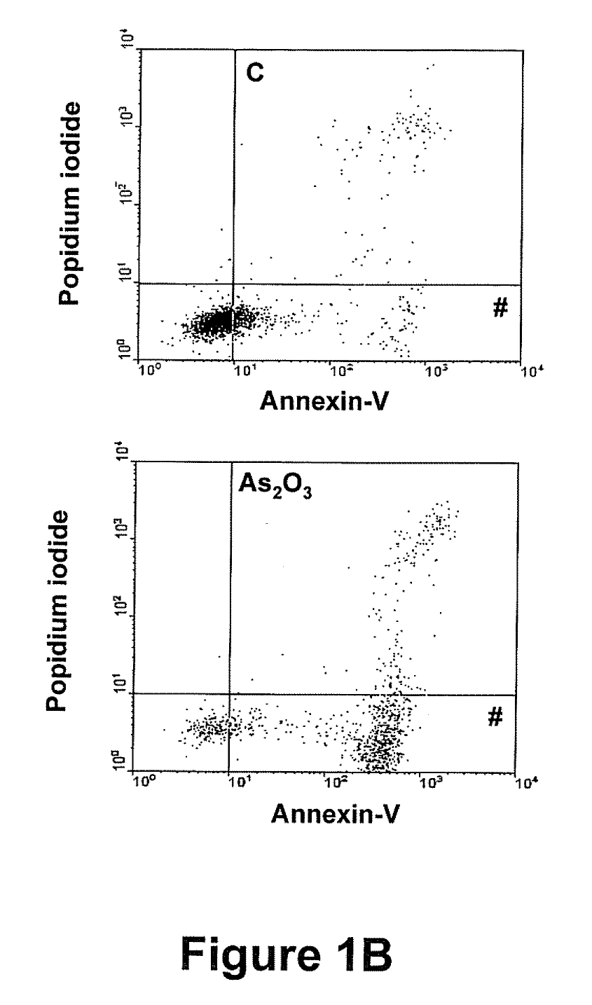

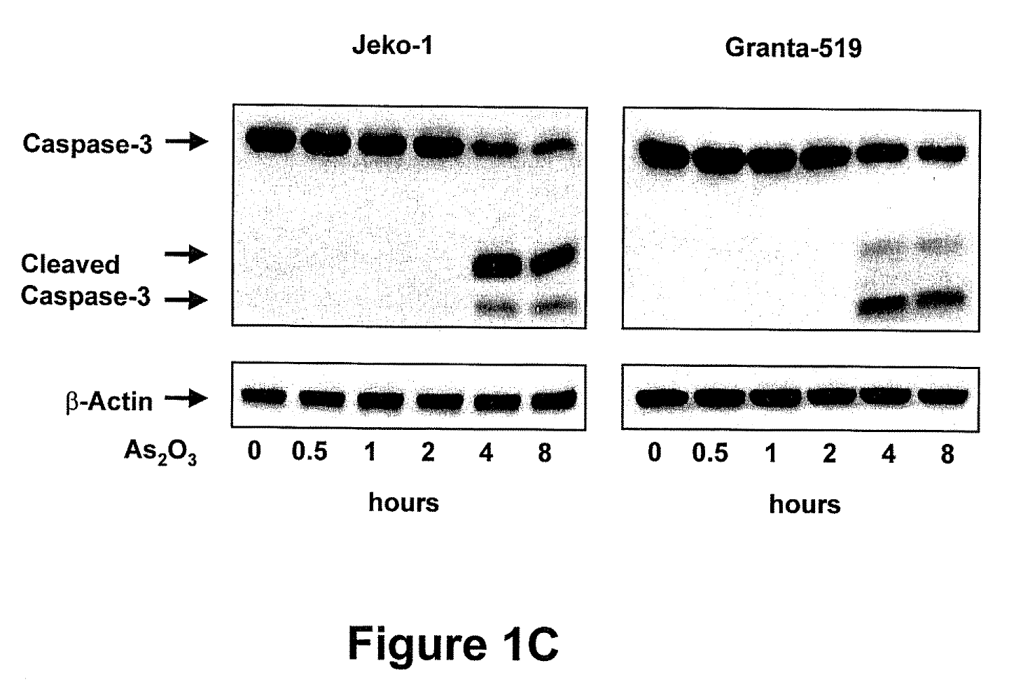

As₂O₃ induced dose and time dependent apoptosis in MCL cells. MTT test showed that As₂O₃ induced a dose-dependent cytotoxicity in Jeko-1 and Granta-519 cells (FIG. 1A). Flow cytometric analysis showed that As₂O₃ treatment led to induction of apoptosis (FIG. 1B). Western blot analysis showed that caspase 3 activation was involved in As₂O₃-induced apoptosis (FIG. 1C).

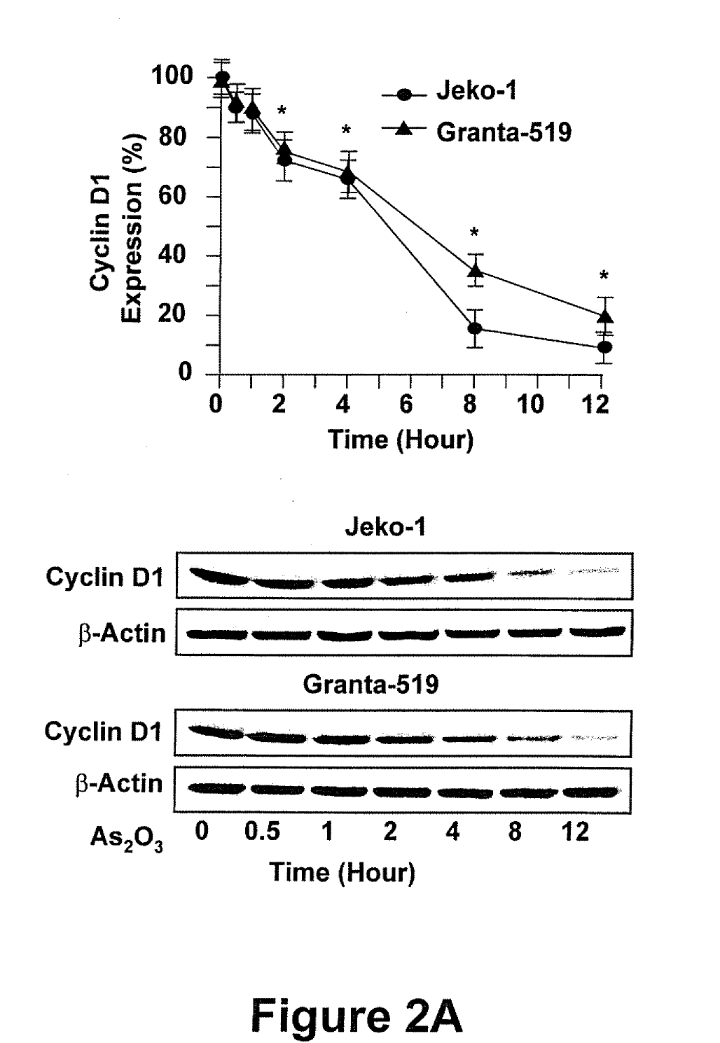

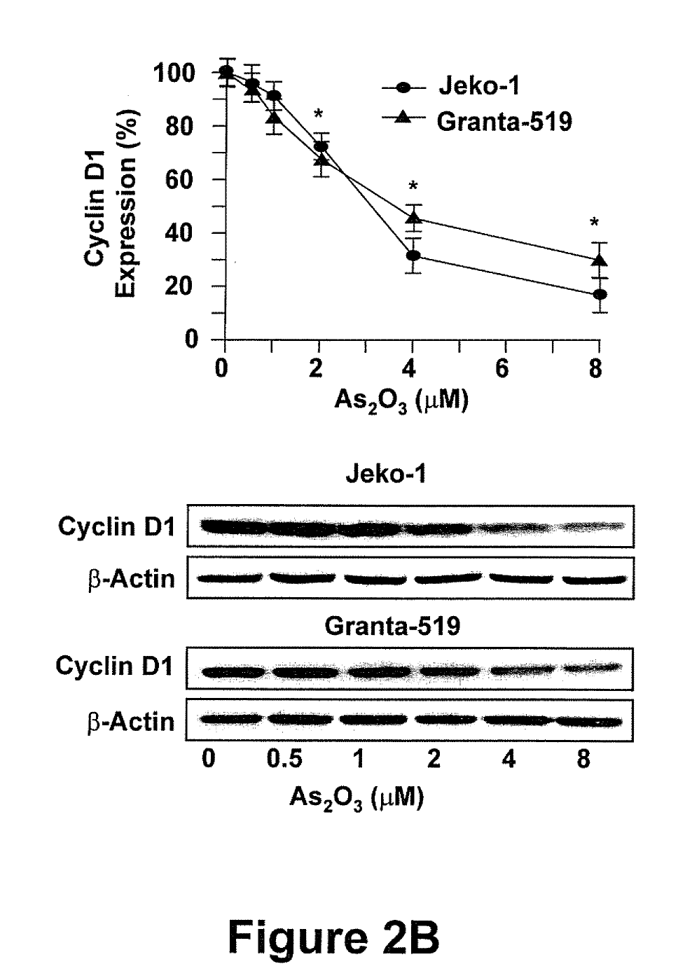

Cyclin D1 was down-regulated in MCL by As₂O₃. To determine the molecular mechanisms of As₂O₃-induced apoptosis in MCL, the expression of cyclin D1 was examined. Western blot analysis showed that As₂O₃-induced a time and dose dependent suppression of cyclin D1 in both cell lines. Treatment with As₂O₃ at 4 □M led to suppression of cyclin D1, first detectable at 2 hours and almost complete at 8-12 hours (FIG. 2A). As₂O₃ suppression of cyclin D1 was also dose-dependent (FIG. 2B).

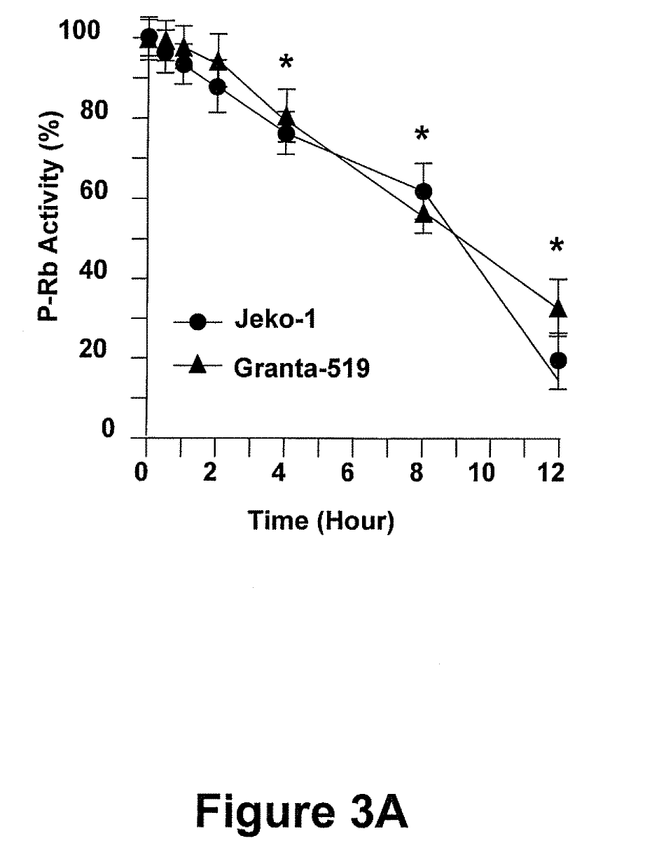

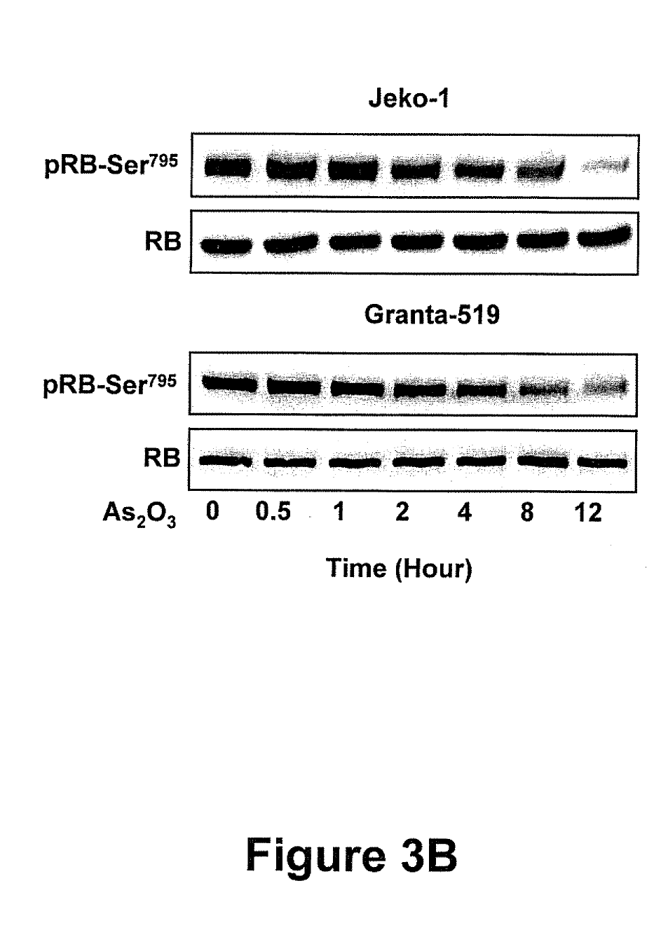

As₂O₃ induced down-regulation of cyclin D1 disrupted its signaling. To investigate if cyclin D1 down-regulation is biologically relevant, RB phosphorylation was investigated. As₂O₃ treatment led to a time dependent decrease in RB phosphorylation, which occurred at a similar time-frame as compared with cyclin D1 down-regulation (FIGS. 3A and B). Cell cycle analysis by flow cytometry showed that there was an increase in the proportion of apoptotic cells.

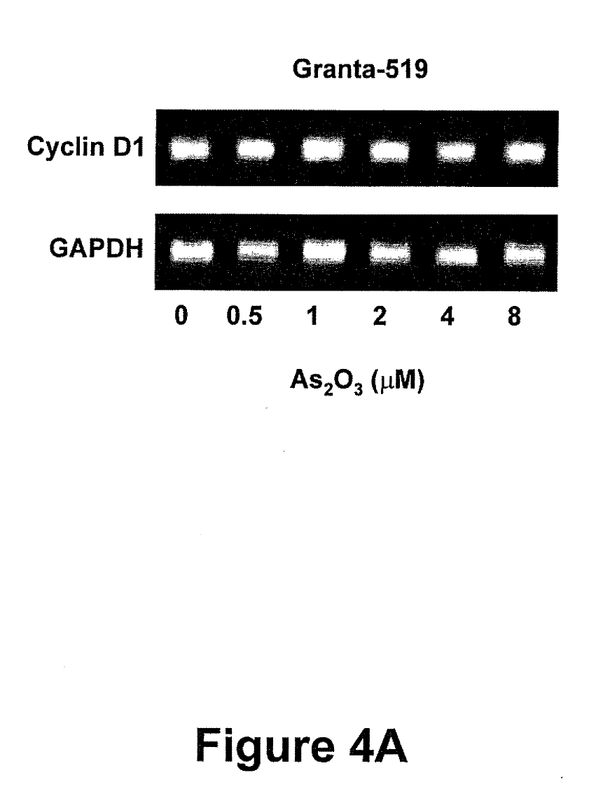

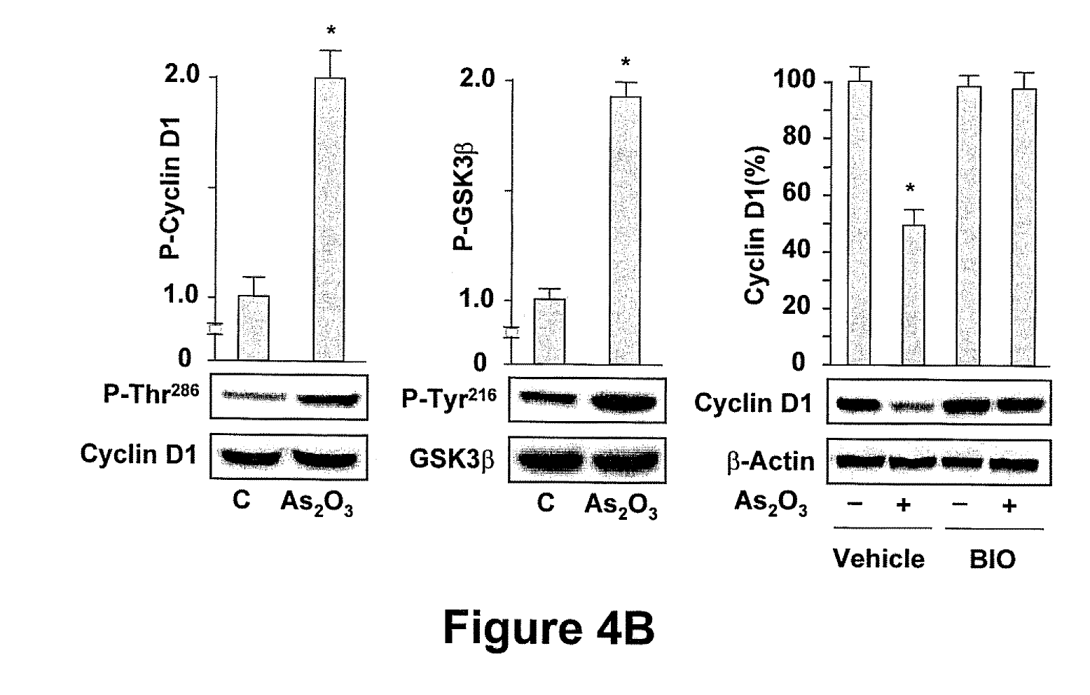

Down-regulation of cyclin D1 by As₂O₃ was post-transcriptional RT-PCR showed that cyclin-D1 gene transcription was unaffected by As₂O₃ treatment of up to 8 μM, suggesting that the down-regulation of cyclin D1 was post-transcriptional (FIG. 4A).

As₂O₃-induced cyclin D1 down-regulation was related to GSK3□ activation. Western blot analysis showed that As₂O₃ treatment resulted in significant increases in cyclin D1 phosphorylation at Thr-286, a prerequisite for cyclin D1 degradation (FIG. 4B). Cyclin D1 phosphorylation by GSK-3β requires prior activation of GSK-3β by phosphorylation at Tyr-216. As₂O₃ treatment in fact significantly increased GSK-3β Tyr-216 phosphorylation, suggesting that GSK-3β might mediate As₂O₃-induced cyclin D1 phosphorylation and hence degradation. To confirm the role of GSK-3β as a mediator of As₂O₃, Jeko-1 cells were pre-incubated with the GSK-3β inhibitor 6-bromoindirubin-3′-oxime (BIO; 10 μM) before As₂O₃ treatment. The results showed that BIO successfully prevented As₂O₃-induced down-regulation of cyclin D1. Collectively, these observations indicated that As₂O₃ down-regulated cyclin D1 post-transcriptionally, probably by increasing its degradation.

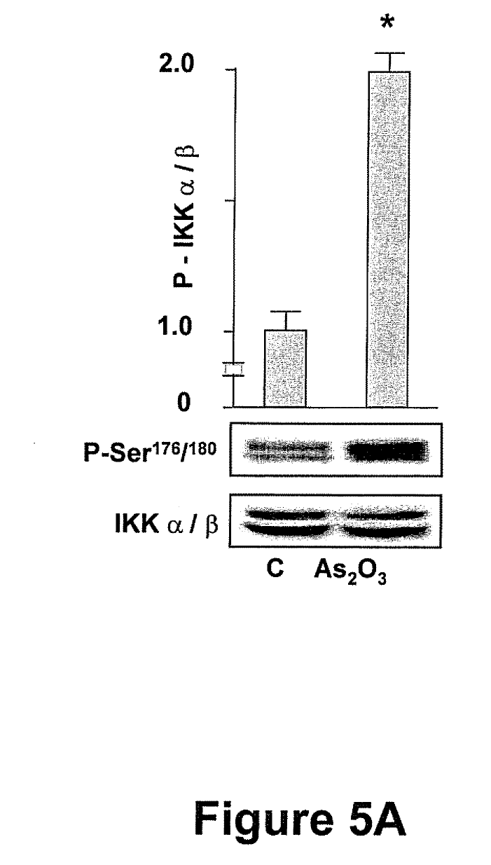

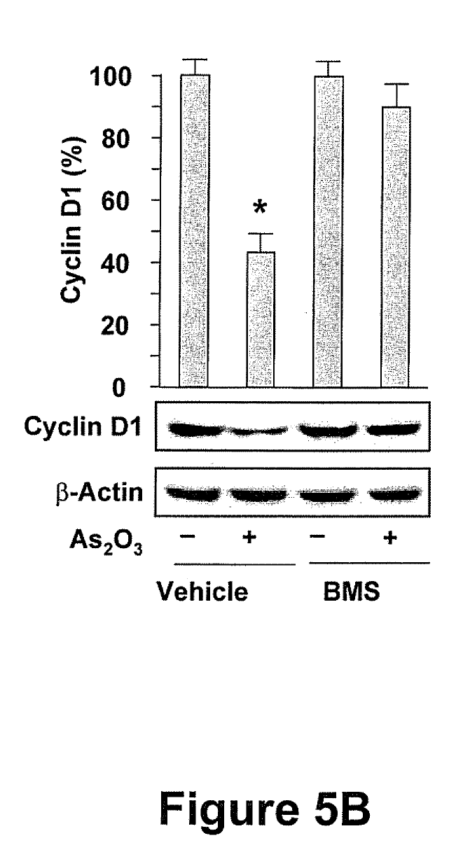

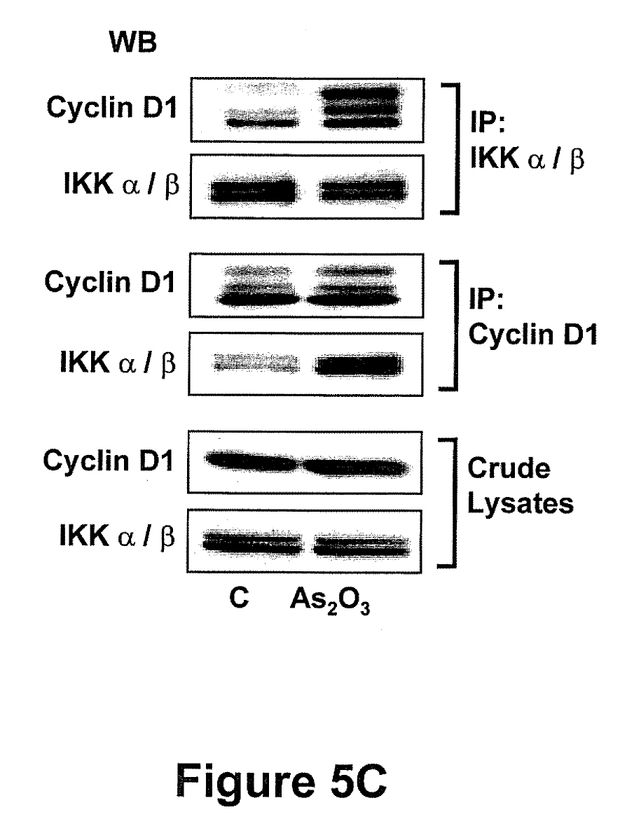

As₂O₃-induced cyclin D1 down-regulation was also dependent on IKKα/β. To determine if IKK was involved in As₂O₃-induced down-regulation of cyclin D1, IKKα/β phosphorylation at Ser-178/180 was examined. As₂O₃ significantly increased IKKα/β Ser-178/180 phosphorylation, which was required for activation of IKKα/β (FIG. 5A). Pre-treatment with the IKKα/β inhibitor BMS-345541 (BMS; 10 μM) significantly prevented As₂O₃-induced cyclin D1 down-regulation, suggesting that IKKα/β was a molecular mediator of As₂O₃ (FIG. 5B) Immunoprecipitation with an anti-IKKα/β antibody showed that cyclin D1 bound IKKα/β. Similarly, when cyclin D1 was immunoprecipitated, IKKα/β was also confirmed to co-immunoprecipitate (FIG. 5C). These results confirmed that As₂O₃ activated IKKα/β, which participated in the down-regulation of cyclin D1.

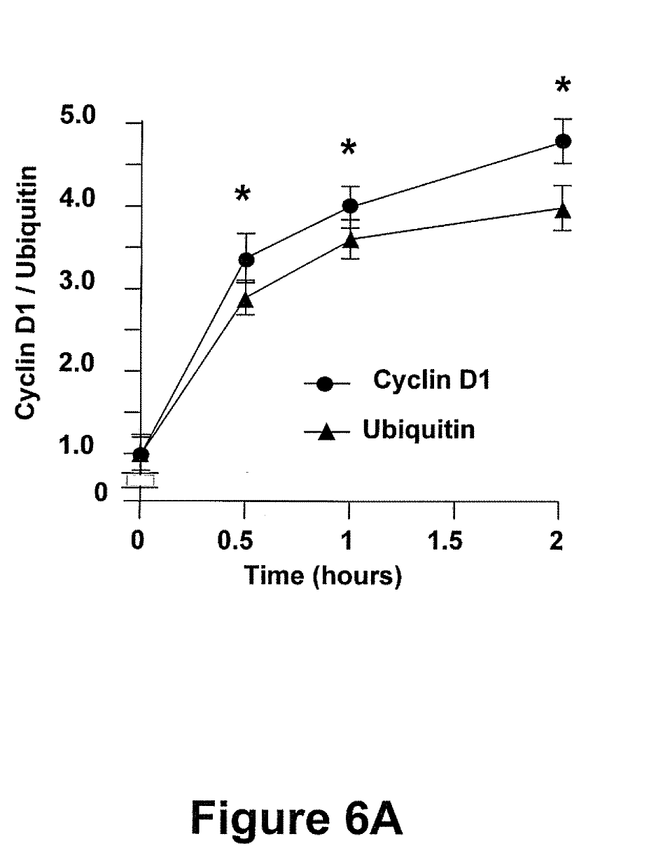

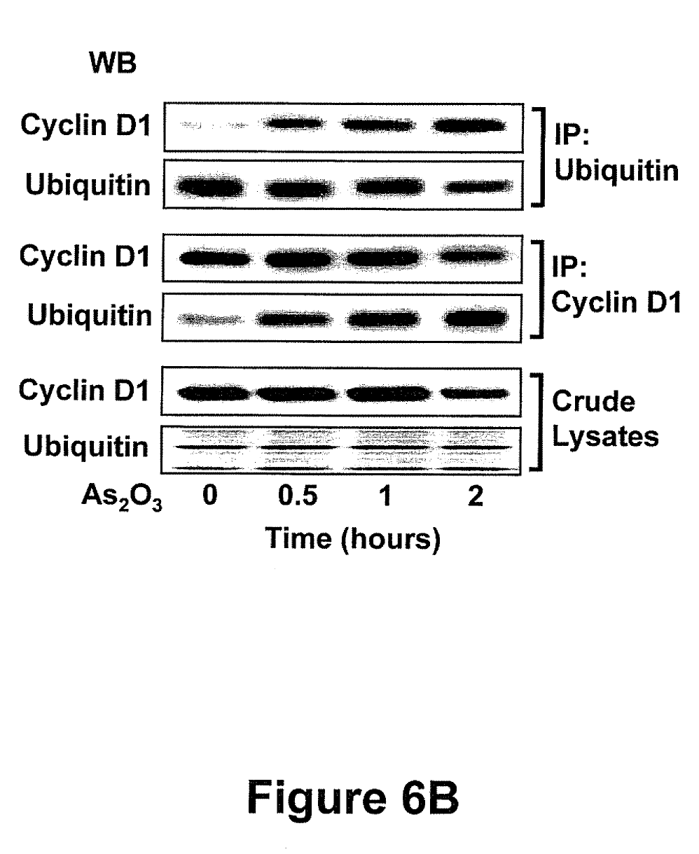

As₂O₃ promoted cyclin D1 ubiquitination. To study if As₂O₃-induced cyclin D1 down-regulation was mediated via ubiquitination, immunoprecipitation experiments were performed on lystaes from Jeko-1 cells treated with As₂O₃. Immunoprecipitation with an anti-ubiquitin antibody showed a time-dependent increase in bound cyclin D1 (FIGS. 6A and B). Similarly, lysates immunoprecipitated with an anti-cyclin D1 antibody also showed a time dependent increase in bound ubiquitin. These results showed that As₂O₃ promoted cyclin D1 ubiquitination, confirming that As₂O₃-induced GSK-3β and IKKα/β activation was biologically relevant.

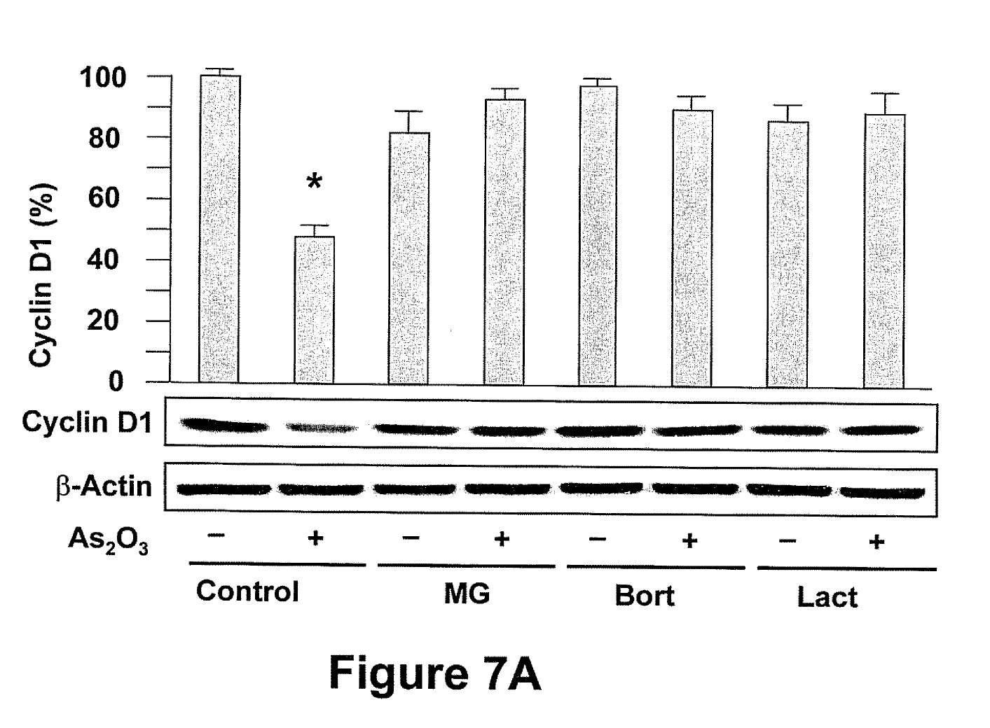

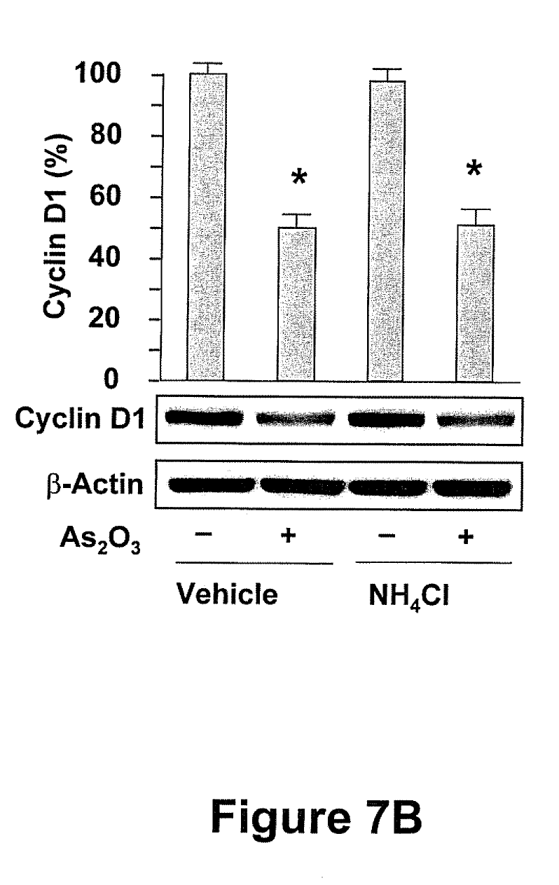

As₂O₃ induced cyclin D1 degradation in 26S and 20S proteasomes but not lysosomes. Pre-incubation of Jeko-1 cells with the 26S and 20S proteosome inhibitors MG132 (30 μM), bortezimab (10 μg/ml) and lactacystin (10 μM) attenuated As₂O₃induced cyclin D1 down-regulation (FIG. 7A). However, pre-incubation with the lysosomal inhibitor ammonium chloride (NH₄Cl) had no effect on As₂O₃-induced down-regulation of cyclin D1 (FIG. 7B). The results confirmed that As₂O₃ down-regulated cyclin D1 by promoting its ubiquitination, hence targeting it to the proteosome for degradation.

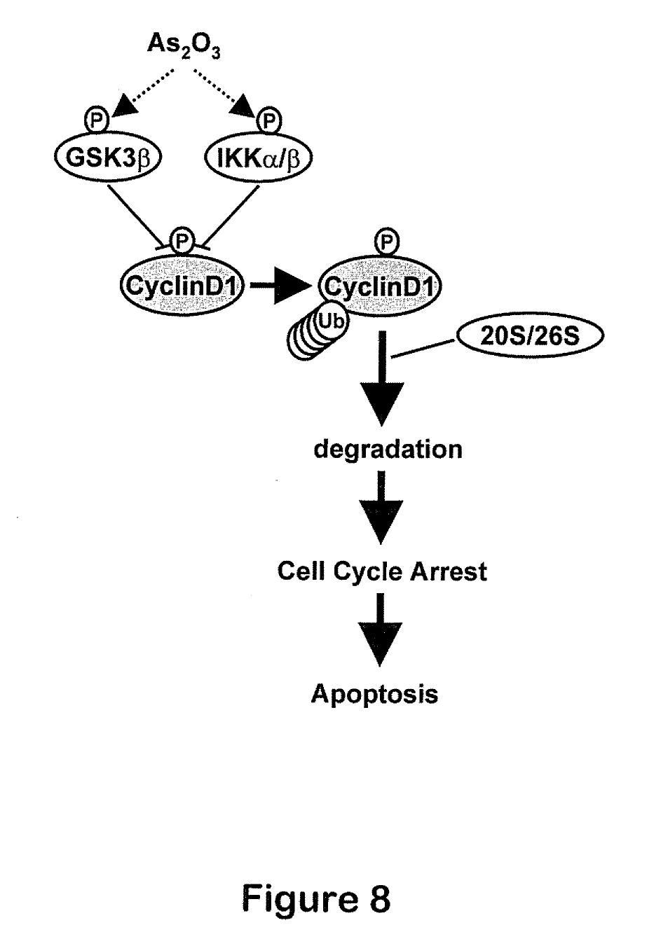

Overall model An overall model of the action of As₂O₃ on MCL is shown in FIG. 8.

### EXAMPLE 8 OF ORAL-As₂O₃ IN THE CLINICAL TREATMENT OF PATIENTS WITH REFRACTORY AND RELAPSED MCL THAT OVER-EXPRESSED CYCLIN D1

Patients, Consenting patients with relapsed or refractory B-cell lymphomas, and an ECOG performance status of <2 were recruited All patients gave informed consent, and the treatment was approved by the institute review board of Queen Mary Hospital.

### Example 9 OF ORAL -As₂O₃ IN THE CLINICAL TREATMENT OF PATIENTS REFRACTORY AND RELAPSED MCL THAT OVER-EXPRESSED CYCLIN D1

Treatment. Treatment was initiated with oral-As₂O₃ (10 mg/day for patients below 70 years old with normal renal function; 5 mg/day for patients over 70 years old, or with impaired renal function), ascorbic acid (AA, 1 g/day) and chlorambucil (4 mg/day) as outpatients until disease response or progression was documented. In patients with bulky disease, debulking with VPP (vincristine 2 mg/day×1, prednisolone 30 mg/day×14 and procarabzine 50-100 mg/day×14) was used. After maximum response was achieved, chlorambucil was taken off and a maintenance regimen of As₂O₃ (5-10 mg/day) and AA (1 g/day) was given for two weeks every 2 months for a planned two years. Responses were classified according to standard NCl criteria,¹⁶ and monitored by regular physical examination, marrow and blood assessment, and computerized tomographic scans.

### RESULTS OF EXAMPLES 8-9 OF ORAL-As₂O₃ IN THE CLINICAL TREATMENT OF PATIENTS WITH REFRACTORY AND RELAPSED MCL THAT OVER-EXPRESSED CYCLIN D1

Characteristics of patients with MCL. Table 1 shows results of the clinical use of oral-As₂O₃ in patients with refractory or relapsed mantle cell lymphoma that over-expressed cyclin D1. The results showed an overall response rate of 64%. Four patients achieved complete remission (CR), whereas two patients achieved complete remission unconfirmed. Of the fourteen patients treated (Table 1), eleven had advanced relapses (R) (R2, n=5; R3, n=4; R4, n=2). Three patients treated in R1 had advanced age (76, 77 and 90 years). All but two patients had received an anthracycline based multi-agent chemotherapy. Other previous treatment included rituximab (n=8), autologous hematopoietic stem cell transplantation (HSCT) (n=3), and bortezomib (n=1). Other poor prognostic indicators included marrow infiltration (n=11) and extensive extranodal involvement (n=9), so that 12/14 (86%) cases had stage IV disease. The median time from initial diagnosis to As₂O₃ treatment was 33 (8-85) months

TABLE 1
Clinicopathologic features and treatment outcome of 14 patients with relapsed or refractory MCL

| | | Initial disease | | | Current relapse | | | | | Outcome and |
|---|---|---|---|---|---|---|---|---|---|---|
| | | stage | sites | Previous treatment | Time* | No | Sites | Total As₂O₃ | response | survival |
| 1 | M/69 | III | Colon, abdomen | FND × 6, COPP × 6 | 56 m | 2 | Cervical | 140 mg | CR | off Rx, 28 m+. |
| 2 | M/63 | IV | BM, generalized LN | R-CEOP × 6, IMVP × 6 | 11 m | 2 | BM, cervical | 160 mg | CR | on Rx, 13 m+. |
| 3 | M/65 | IV | BM, mesentery, | FND × 7, IMVP × 2, | 85 m | 3 | Eye | 120 mg | CR | on Rx, 17 m+. |
| | | | generalized LN | R-DHAP × 8 | | | | | | |
| 4 | F/77 | IV | Pleura, | Clb | 33 m | 1 | Groin, jaw | 140 mg | CR | R2 at 16 m, |
| | | | generalized LN | | | | | | | CR again with |
| | | | | | | | | | | As₂O₃ + Clb |
| 5 | M/70 | III | Generalized LN | COPP × 2, IMVP × 6, Clb | 85 m | 4 | Cervical, abdomen | 250 mg | CRu | R5 at 20 m, on |
| | | | | | | | | | | As₂O₃ + Clb |
| 6 | M/76 | IV | BM, generalized LN | CEOP × 7 | 19 m | 1 | BM, leukemic, eyes, | 210 mg* | CRu | on Rx, 8 m+ |
| | | | | | | | generalized LN | | | |
| 7 | M/58 | IV | BM, generalized LN | CEOP × 6, R-ESHAP × 6 | 18 m | 2 | Generalized skin | 140 mg | PR | On Rx, 3 m+ |
| 8 | M/81 | IV | BM, leukemic, | CHOP × 6, ChlVPP × 2 | 18 m | 2 | BM, LN, liver, spleen, | 300 mg* | PR | died at 16 m |
| | | | liver, spleen | | | | leukemic | | | |
| 9 | M/51 | IV | Generalized | CVAD × 7, CEOP × 2, | 25 m | 4 | BM, LN, scalp | NA* | Static | on Rx, 8 m+ |
| | | | LN, spleen, BM, | R-DHAP × 3, Thal | | | | | | |
| | | | scalp, eye | | | | | | | |
| 10 | F/76 | IV | General LN, | R-COPP × 6 | 12 m | 2 | BM, LN | 160 mg* | PR | died at 6 m |
| | | | BM, scalp | | | | | | | |
| 11 | M/90 | IV | BM, leukemic | Clb | 8 m | 1 | BM, leukemic | NA | NR | died at 4 m |
| 12 | M/54 | IV | Generalized LN, | CEOP × 6, DHAP × 1, | 36 m | 3 | BM, generalized LN, | NA | Static | died at 17 m |
| | | | BM, gut, liver, | NOPP × 5, Clb | | | spleen | | | |
| | | | spleen, leukemic | | | | | | | |
| 13 | F/57 | IV | Generalized LN, | CEOP × 6, DHAP × | 36 m | 3 | BM, LN | NA | NR | died at 1 m |
| | | | BM, spleen | 4, R-BVP × 3 | | | | | | |
| 14 | M/63 | IV | Generalized LN, | CEOP × 6, AHSCT, | 72 m | 3 | BM, generalized LN | NA | NR | died at 1 m |
| | | | pleura, BM | R-DHAP × 6, | | | | | | |
| | | | | Thal, velcade, FND | | | | | | |

M: male;
F: female;
LN: lymphadenopathy;
BM: bone marrow;
m: months;
R: rituximab;
CEOP: cyclophospamide, epirubicin, vincristine, predniolone
FND: fludarabine, mitoxantrone, dexamethasone;
DHAP: cisplatinum, cytosine arabinoside, dexamethasone;
Thal: thalidomide
ChlvPP: chlorambucil, vincristine, procarbazine, prednisolone;
COPP: cyclophosphamide, vincristine, procarbazine, prednisolone;
NOPP: mitoxantrone; vincristine, procarbazine, prednisolone;
BVP; bleomycin, vinblastine, prednisolone;
AHSCT: autologous hematopoietic stem cell transplantation
Clb: chlorambucil;
NA: not available;
CR: complete remission;
CRu: complete remission (unconfirmed);
PR: partial remission;
NR; no response

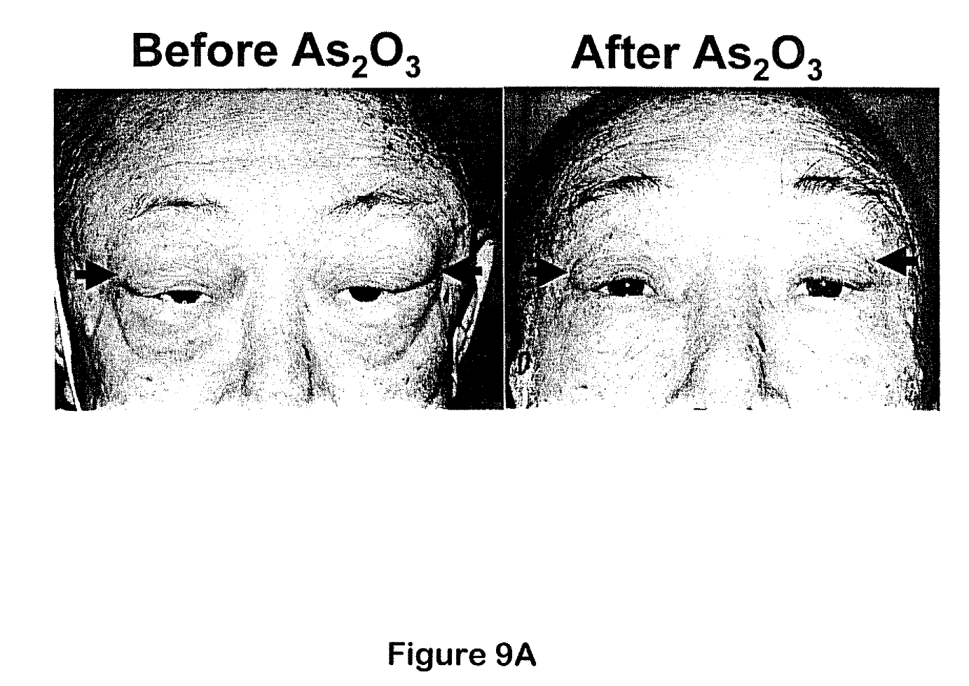

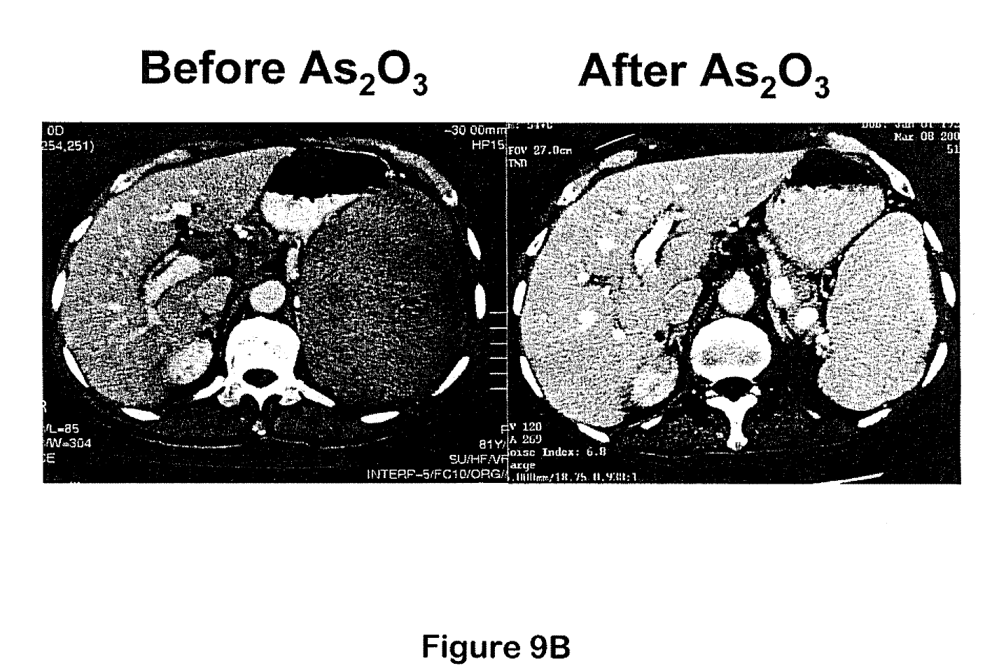

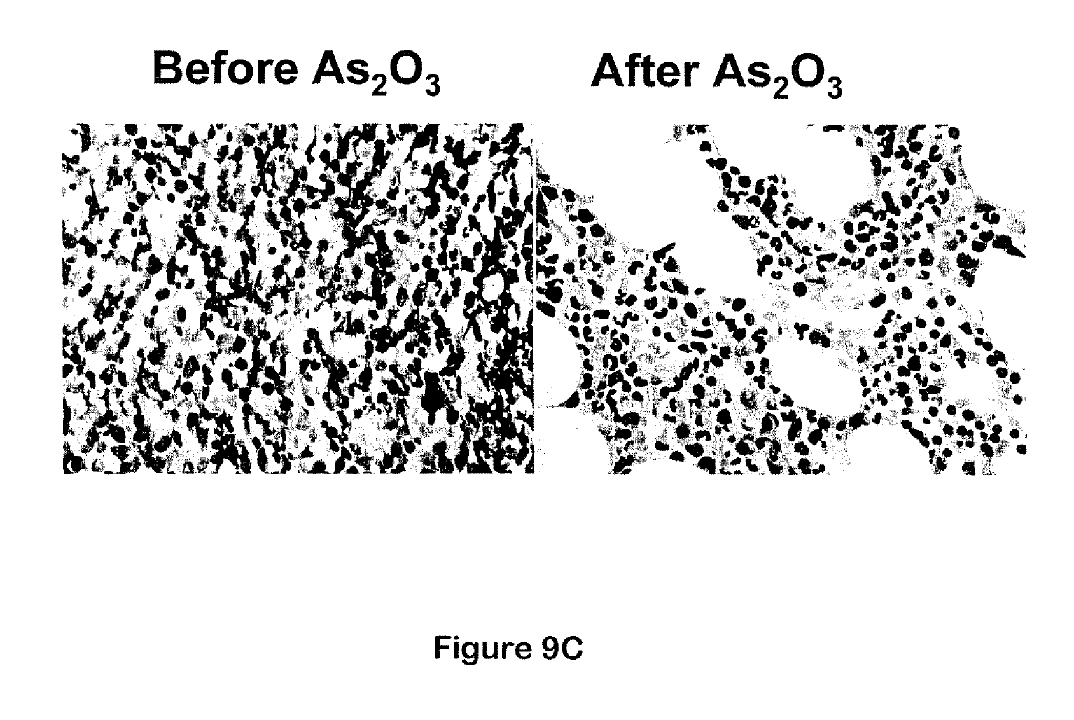

Treatment response. Nine patients responded, giving an OR rate of 64%. Four patients (cases 1-4) achieved CR. Two patients (cases 5, 6) achieved unconfirmed CR (CRu). They had become asymptomatic without any detectable superficial diseases (FIG. 9A). Marrow and peripheral blood involvement was also cleared. However, small residual internal lymph nodes remained. These lymph nodes were negative on gallium scan and had remained static in size. Three patients had partial responses (PR) with >50% reduction in the size of assessable lymph nodes (FIGS. 9B and C).

Outcome. Of the four patients with CR, one had relapsed at 16 months. She achieved a CR3 again with daily As₂O₃ and resumption of chlorambucil. Two patients were still on maintenance As₂O₃+AA treatment, while one had completed the planned two years of treatment. Of the two patients with CRu, one patient had relapsed at 20 months. He achieved CR5 again with As₂O₃ and chlorambucil therapy. For the three patients with PR, one patient developed progressive disease while on maintenance therapy 12 months later and died of refractory lymphoma. Two defaulted treatment and both relapsed.

Toxicity. Significant (W.H.O grade 3-4) neutropenia and thrombocytopenia was observed in 7 patients. These patients had previously multiple chemotherapy, or autologous HSCT. The neutropenia responded to hematopoietic growth factors. No significant sepsis or bleeding were observed. Other side effects included fever (n=7), herpes zoster reactivation (n=3), fluid accumulation (n=2), nausea (n=3) and headache (n=2). No significant QT prolongation or arrhythmia was observed. Five patients did not report any side effects at all.

### REFERENCES

1. Sherr C J. Cancer cell cycles. Science 1996;274:1672-7.
2. Sherr C J, McCormick F. The RB and p53 pathways in cancer. Cancer Cell 2002;2:103-112.
3. Guo Y, Stacey D W, Hitomi M. Post-transcriptional regulation of cyclin D1 expression during G2 phase. Oncogene 2002;21:7545-56.
4. Guo Y, Yang K, Harwalkar J, Nye J M, Mason D R, Garrett M D, Hitomi M, Stacey D W. Phosphorylation of cyclin D1 at Thr 286 during S phase leads to its proteasomal degradation and allows efficient DNA synthesis. Oncogene 2005;24:2599-612.
5. Diehl J A, Zindy F, Sherr C J. Inhibition of cyclin D1 phosphorylation on threonine-286 prevents its rapid degradation via the ubiquitin-proteasome pathway. Genes Dev 1997;11:957-72.
6. Diehl J A, Cheng M, Roussel M F, Sherr C J. Glycogen synthase kinase-3beta regulates cyclin D1 proteolysis and subcellular localization. Genes Dev 1998;12:3499-511.
7. Alt J R, Cleveland J L, Hannink M, Diehl J A. Phosphorylation-dependent regulation of cyclin D1 nuclear export and cyclin D1-dependent cellular transformation. Genes Dev 2000;14:3102-14.
8. Kwak Y T, Li R. Becerra C R, Tripathy D, Frenkel E P, Verma U N, IkappaB kinase alpha regulates subcellular distribution and turnover of cyclin D1 by phosphorylation. J Biol Chem 2005;280:33945-52.
9. Swerdlow S H, Berger F, Isaacson P I, Muller-Hermelink H K, Nathwani B N, Piris M A, Harris N L. Mantle cell lymphoma. In Jaffe E S, Harris N L, Styein H, Vardiman J W (Eds). World Health Organization classification of tumours. Pathology and genetics of tumours of hematopoietic and lymphoid tissues. Lyon, France, IARC Press, 2001, 168-170.
10. The Non-Hodgkin's Lymphoma Classification Project. A clinical evaluation of the International Lymphoma Study Group classification of non-Hodgkin's lymphoma. Blood 1997;89:3909-3918.
11. Tsujimoto Y, Yunis J, Onorato-Showe L, Erikson J, Nowell P C, Croce C M. Molecular cloning of the chromosomal breakpoint of B-cell lymphomas and leukemias with the t(11;14) chromosome translocation. Science 1984;224:1403-6.
12. Tsujimoto Y, Jaffe E, Cossman J, Gorham J, Nowell P C, Croce C M. Clustering of breakpoints on chromosome 11 in human B-cell neoplasms with the t(11;14) chromosome translocation. Nature 1985;315:340-3.
13. Witzig T E. Current treatment approaches for mantle-cell lymphoma. J Clin Oncol 2005;23:6409-14.
14. Lenz G, Dreyling M, Hoster E, Wormann B, Duhrsen U, Metzner B, Eimermacher H, Neubauer A, Wandt H, Steinhauer H, Martin S, Heidemann E, Aldaoud A, Parwaresch R, Hasford J, Unterhalt M, Hiddemann W. Immunochemotherapy with rituximab and cyclophosphamide, doxorubicin, vincristine, and prednisone significantly improves response and time to treatment failure, but not long-term outcome in patients with previously untreated mantle cell lymphoma: results of a prospective randomized trial of the German Low Grade Lymphoma Study Group (GLSG). J Clin Oncol 2005;23:1984-92.
15. Forstpointner R, Dreyling M, Repp R, Hermann S, Hanel A, Metzner B, Pott C, Hartmann F, Rothmann F, Rohrberg R, Bock H P, Wandt H, Unterhalt M, Hiddemann W; German Low-Grade Lymphoma Study Group. The addition of rituximab to a combination of fludarabine, cyclophosphamide, mitoxantrone (FCM) significantly increases the response rate and prolongs survival as compared with FCM alone in patients with relapsed and refractory follicular and mantle cell lymphomas: results of a prospective randomized study of the German Low-Grade Lymphoma Study Group. Blood 2004;104:3064-71.
16. Romaguera J E, Fayad L, Rodriguez M A, Broglio K R, Hagemeister F B, Pro B, McLaughlin P, Younes A, Samaniego F, Goy A, Sarris A H, Dang N H, Wang M, Beasley V, Medeiros L J, Katz R L, Gagneja H, Samuels B I, Smith T L, Cabanillas F F. High rate of durable remissions after treatment of newly diagnosed aggressive mantle-cell lymphoma with rituximab plus hyper-CVAD alternating with rituximab plus high-dose methotrexate and cytarabine. J Clin Oncol 2005;23:7013-23.
17. Goy A, Younes A, McLaughlin P, Pro B, Romaguera J E, Hagemeister F, Fayad L, Dang N H, Samaniego F, Wang M, Broglio K, Samuels B, Gilles F, Sarris A H, Hart S, Trehu E, Schenkein D, Cabanillas F, Rodriguez A M. Phase II study of proteasome inhibitor bortezomib in relapsed or refractory B-cell non-Hodgkin's lymphoma. J Clin Oncol 2005;23:667-75.
18. O'Connor O A, Wright J, Moskowitz C, Muzzy J, MacGregor-Cortelli B, Stubblefield M, Straus D, Portlock C, Hamlin P, Choi E, Dumetrescu O, Esseltine D, Trehu E, Adams J, Schenkein D, Zelenetz A D. Phase II clinical experience with the novel proteasome inhibitor bortezomib in patients with indolent non-Hodgkin's lymphoma and mantle cell lymphoma. J Clin Oncol 2005;23:676-84.
19. Kaufmann H, Raderer M, Wohrer S, Puspok A, Bankier A, Zielinski C, Chott A, Drach J. Antitumor activity of rituximab plus thalidomide in patients with relapsed/refractory mantle cell lymphoma. Blood 2004;104;2269-71.
20. Witzig T E, Geyer S M, Ghobrial I, Inwards D J, Fonseca R. Kurtin P, Ansell S M, Luyun R, Flynn P J, Morton R F, Dakhil S R, Gross H, Kaufmann S H. Phase II trial of single-agent temsirolimus (CCI-779) for relapsed mantle cell lymphoma. J Clin Oncol 2005;23:5347-56.
21. Kwong Y L. Arsenic trioxide in the treatment of haematological malignancies. Expert Opin Drug Saf 2004;3:589-97.
22. Kumana C R, Au W Y, Lee N S, Kou M, Mak R W, Lam C W, Kwong Y L. Systemic availability of arsenic from oral arsenic-trioxide used to treat patients with hematological malignancies. Eur J Clin Pharmacol 2002;58:521-6.
23. Au W Y, Kumana C R, Kou M, Mak R, Chan G C, Lam C W, Kwong Y L. Oral arsenic trioxide in the treatment of relapsed acute promyelocytic leukemia. Blood 2003;102:407-8.
24. Siu C W, Au W Y, Yung C, Kumana C R, Lau C P, Kwong Y L, Tse H F. Effects of oral arsenic trioxide therapy on QT intervals in patients with acute promyelocytic leukemia: implications for long-term cardiac safety. Blood 2006;108;103-6.
25. Kauffmann-Zeh A, Rodriguez-Viciana P, Ulrich E, Gilbert C, Coffer P, Downward J, Evan G. Suppression of c-Myc-induced apoptosis by Ras signalling through PI(3)K and PKB. Nature 1997;385:544-8.
26. Vanhaesebroeck B, Leevers S J, Panayotou G, Waterfield M D. Phosphoinositide 3-kinases: a conserved family of signal transducers. Trends Biochem Sci 1997;22:267-72.
27. Cross D A, Alessi D R, Cohen P, Andjelkovich M, Hemmings B A. Inhibition of glycogen synthase kinase-3 by insulin mediated by protein kinase B. Nature 1995;378:785-9.
28. Hughes K, Nikolakaki E, Plyte S E, Totty N F, Woodgett J R. Modulation of the glycogen synthase kinase-3 family by tyrosine phosphorylation. EMBO J 1993;12:803-8.
29. Cole A, Frame S, Cohen P. Further evidence that the tyrosine phosphorylation of glycogen synthase kinase-3 (GSK3) in mammalian cells is an autophosphorylation event. Biochem J 2004;377:249-55.
30. Kim L, Liu J, Kimmel A R. The novel tyrosine kinase ZAK1 activates GSK3 to direct cell fate specification. Cell 1999;99:399-408.
31. Lesort M, Jope R S, Johnson G V. Insulin transiently increases tau phosphorylation: involvement of glycogen synthase kinase-3beta and Fyn tyrosine kinase. J Neurochem 1999:72:576-84.
32. Fan G, Ballou L M, Lin R Z. Phospholipase C-independent activation of glycogen synthase kinase-3beta and C-terminal Src kinase by Galphaq. J Biol Chem December 2003;278:52432-6.
33. Hartigan J A, Xiong W C, Johnson G V. Glycogen synthase kinase 3beta is tyrosine phosphorylated by PYK2. Biochem Biophys Res Commun 2001;284:485-9.
34. Sayas C L, Ariaens A, Ponsioen B, Moolenaar W H. GSK-3 is activated by the tyrosine kinase Pyk2 during LPA1-mediated neurite retraction. Mol Biol Cell 2006;17:1834-44.
35. Hartigan J A, Johnson G V. Transient increases in intracellular calcium result in prolonged site-selective increases in Tau phosphorylation through a glycogen synthase kinase 3beta-dependent pathway. J Biol Chem 1999;274:21395-401.
36. Ling L, Cao Z, Goeddel D V. NF-kappaB-inducing kinase activates IKK-alpha by phosphorylation of Ser-176. Proc Nati Acad Sci U S A. 1998;95:3792-7.
37. Burke J R, Pattoli M A, Gregor K R, Brassil P J, MacMaster J F, Mcintyre K W, Yang X, lotzova V S, Clarke W, Strnad J, Qiu Y, Zusi F C. BMS-345541 is a highly selective inhibitor of I kappa B kinase that binds at an allosteric site of the enzyme and blocks NF-kappa B-dependent transcription in mice. J Biol Chem 2003;278:1450-6.
38. Malinin N L, Boldin M P, Kovalenko A V, Wallach D. MAP3K-related kinase involved in NF-kappaB induction by TNF, CD95 and IL-1. Nature 1997;385:540-4.
39. Hicke L. Protein regulation by monoubiquitin. Nat Rev Mol Cell Biol 2001;2:195-201.
40. Sternsdorf T, Puccetti E, Jensen K, Hoelzer D, Will H, Ottmann O G, Ruthardt M, PIC-1/SUMO-1-modified PML-retinoic acid receptor alpha mediates arsenic trioxide-induced apoptosis in acute promyelocytic leukemia. Mol Cell Biol 1999;19:5170-8.
41. Lallemand-Breitenbach V, Zhu J. Puvion F, Koken M, Honore N, Doubeikovsky A, Duprez E, Pandolfi P P, Puvion E, Freemont P, de The H. Role of promyelocytic leukemia (PML) sumolation in nuclear body formation, 11S proteasome recruitment, and As2O3-induced PML or PML/retinoic acid receptor alpha degradation. J Exp Med 2001;193:1361-71.

What is claimed is:

1. A method for inhibiting cyclin D1 production in a cell, comprising contacting the cell with an amount of arsenic trioxide effective to inhibit cyclin D1 production therein.

2. A method according to claim 1, wherein the cell is a cancer cell.

3. A method according to claim 2, wherein the cell is a human mantel cell lymphoma cell.

4. A method according to claim 2, wherein the cancer cell resides within a patient and the arsenic trioxide is administered orally.

5. A method according to claim 4, wherein the cancer cell is a human female genital tract cancer, a digestive tract cancer, or a malignant lymphoma.

6. A method for inhibiting growth of mantle cell lymphoma (MCL) in a subject afflicted thereby, comprising administering to the subject an MCL growth inhibiting amount of arsenic trioxide in a pharmaceutically acceptable vehicle.

7. A method according to claim 2, wherein the arsenic trioxide is administered orally.

8. A method according to claim 2, wherein MCL growth is inhibited by preventing overexpression of cyclin D1.

9. A method according to claim 3, wherein MCL growth is inhibited by preventing overexpression of cyclin D1.
